(19) United States
(12) Patent Application Publication
(10) Pub. No.: US 2007/0112076 A1
(43) Pub. Date: May 17, 2007

(54) Methods and materials for treating retinopathy

(75) Inventor: Jena Steinle, Carbondale, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: SOUTHERN ILLINOIS UNIVERSITY, Carbondale, IL (US)

(21) Appl. No.: 11/599,641

(22) Filed: Nov. 15, 2006

Related U.S. Application Data
(60) Provisional application No. 60/737,161, filed on Nov. 16, 2005.

(51) Int. Cl. A61K 31/138

(52) U.S. Cl. 514/651

(57) ABSTRACT

The present invention is directed to topical ophthalmic compositions and methods for treating retinopathy.

### CROSS REFERENCE TO RELATED APPLICATION

This application claims the benefit of Provisional U.S. Patent Application No. 60/737,161, filed Nov. 16, 2005, the entirety of which is expressly incorporated herein by reference.

### BACKGROUND

1. Field

The present invention is generally directed to methods and materials for treatment of retinopathy, including diabetic retinopathy and age-related macular degeneration.

2. Background of the Related Technology

Diabetes affected 15.7 million people in the United States in 2000, and this number is expected to double over the next 25 years. A major complication of diabetes is diabetic retinopathy, which can occur in up to 50 percent of patients with type I diabetes. Diabetic retinopathy results from damage to the small blood vessels and neural cells in the retina and as it progresses can lead to blindness. Diabetic retinopathy is the leading cause of blindness in working age adults. In type I diabetes, retinopathy can develop within 7 years after disease onset, placing patients with significant visual defects in the twenties through forties, prime working years

The principal characteristics of diabetic retinopathy are thickening of the basement membrane, loss of pericytes, abnormal proliferation of endothelial cells, and the formation of microaneurysms, leading to neovascularization. Diabetes can also damage the retinal pigment epithelium. The exact causes of these complications of diabetes have not been determined, although it has been suggested that inflammation may play a significant role in the pathogenesis of retinopathy in rats.

Diabetes also significantly affects sympathetic nerves. While it is clear that diabetes has negative effects on sympathetic nerves in the heart and other organs, the potential role of sympathetic nerves in diabetic retinopathy has not been elucidated. Sympathetic denervation has been shown to result in vascular remodeling in the outer retina (Steinle et al., Rat. Exp Eye Res, 2002, 74:761-8). Similar vascular changes have been shown to occur following blockade of the beta-adrenergic system (Steinle and Smith, Br J Pharmacol, 2002, 136:730-4).

The currently available treatments for diabetic retinopathy involve slowing the loss of vision with intravitreal injections of vascular endothelial cell growth factor inhibitors, which are in the experimental phase, or laser therapy to photocoagulate the newly-growing blood vessels. Both of these treatments are used when vision is already compromised and merely slow the loss of vision. Neither of these treatments will reverse the vision loss that occurs in retinopathy and both involve pain to the patient. The design of treatments aimed at the retina is complicated by the blood-retinal barrier, which regulates the movement of molecules in and out of the retina, and the obstacles to absorption of topically applied drugs, which include clearance of topical solutions via tear flow and the cornea's barrier to absorption.

Thus, a need exists for new treatments that can stop or even reverse the retinopathic changes at the pre-proliferative phase before vision loss occurs. Such treatments ideally are also easily administered and well tolerated by the patient due to the need for long-term therapy.

### SUMMARY OF THE INVENTION

The present invention provides topical ophthalmic compositions comprising a beta adrenergic agonist, and methods of using such compositions for treating retinopathy, including diabetic retinopathy and age-related macular degeneration. In exemplary embodiments of the invention, the agonist is non-selective; in other exemplary embodiments, the agonist is selective for beta-1, beta-2 or beta-3 adrenergic receptor.

One aspect of the invention provides a topical ophthalmic composition, preferably in the form of an aqueous solution or suspension, comprising an amount of a beta adrenergic agonist effective to treat retinopathy, including diabetic retinopathy or age-related macular degeneration.

Another aspect of the invention provides methods of using the topical ophthalmic compositions of the invention to treat diabetic retinopathy or age-related macular degeneration.

The invention also provides for the use of a beta adrenergic agonist in preparation of a medicament for the treatment of diabetic retinopathy or age-related macular degeneration.

Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### BRIEF DESCRIPTION OF THE DRAWINGS

FIG. 1 illustrates number of pericytes in sympathectomized retina vs. normal retina.

FIG. 2 illustrates number of acellular capillaries in sympathectomized retina vs. normal retina.

FIG. 3 illustrates PKA levels in retina of normal or diabetic rates treated with isoproterenol, a beta adrenergic agonist.

FIG. 4 illustrates CREB levels in retina of normal or diabetic rates treated with isoproterenol, a beta adrenergic agonist.

FIG. 5A illustrates iNOS mRNA levels assayed by real-time PCR in sympathectomized retina vs. normal retina. FIGS. 5B-5C show results of a typical Western blot and mean densitometry data, and illustrate iNOS protein expression levels in sympathectomized retina vs. normal retina.

FIG. 6 shows absorbance data from a PGE2 ELISA assay in sympathectomized retina vs. normal retina.

FIGS. 7A-7B show results of a typical Western blot and mean densitometry data, and illustrate PGE2-EP2 receptor protein expression levels in sympathectomized retina vs. normal retina.

FIGS. 8A-8B show results of a typical Western blot and mean densitometry data, and illustrate iNOS expression levels at various time periods after treatment of retinal endothelial cells with isoproterenol in high glucose medium.

FIGS. 9A-9B show results of a typical Western blot and mean densitometry data, and illustrate iNOS expression levels at various time periods after treatment of retinal endothelial cells with isoproterenol in low glucose medium.

FIG. 10A shows absorbance data from a PGE2 ELISA assay and illustrates PGE2 protein levels at various time periods after treatment of retinal endothelial cells with isoproterenol in high or low glucose medium.

FIG. 10B shows mean densitometry data and illustrates PGE2 receptor levels at various time periods after treatment of retinal endothelial cells with isoproterenol in high or low glucose medium.

FIGS. 11A-11B show results of a typical Western blot and mean densitometry data, and illustrate iNOS expression levels at various time periods after treatment of retinal endothelial cells with xamoterol in high glucose medium.

FIGS. 12A-12B show results of a typical Western blot and mean densitometry data, and illustrate iNOS expression levels at various time periods after treatment of retinal endothelial cells with BRL37344 in high glucose medium.

### DESCRIPTION OF THE PREFERRED EMBODIMENTS

The invention provides a topical ophthalmic composition comprising an amount of a beta adrenergic agonist effective to treat retinopathy, including diabetic retinopathy or age-related macular degeneration. The term “treatment” as used herein encompasses both prophylactic and therapeutic treatment.

Preliminary data indicate that superior cervical ganglionectomy, which removes sympathetic innervation to all cranial targets, produces basement membrane thickening, pericyte loss, acellular capillaries, and increased capillary density in the outer nuclear layer of the retina. The data herein show that sympathectomy also results in an upregulation of iNOS and PGE2 protein and mRNA expression of iNOS and PGE2-EP2 receptor subtype in the retina, both of which are markers of inflammation, suggesting that the complications in the retina mediated by sympathectomy may be due to activation of inflammatory mediators. The data herein also show that administration of a beta-adrenergic agonist reduced inflammatory mediator production in retinal endothelial cells, one of the cell types in the retina that are involved in inflammatory cytokine production. Regulation of iNOS appears to be primarily through beta-1 adrenergic receptors, since administration of a beta-1 selective adrenergic agonist provided a reduction in the increased expression of iNOS. The effect of beta-adrenergic agonists on PGE2 levels may be through their effect on other cell types, such as Muller cells, pericytes or glial cells.

Many of these same histopathological and inflammatory changes occur in the streptozotocin (STZ)-treated diabetic rat model. STZ-treatment is also associated with significantly reduced dopamine beta hydroxylase, a marker of sympathetic neurotransmission, in the rat retina, as early as 4 weeks after diabetes onset. These results suggest that loss of sympathetic nerve activity may be involved in a number of the retinal changes noted in pre-proliferative retinopathy. Beta adrenergic agonist therapy is ideally begun as soon as loss of sympathetic neurotransmission is noted, or earlier, to prevent the vascular proliferation which results in vision loss.

The data described herein indicate that loss of sympathetic innervation contributes to vascular, neural and inflammatory features of early diabetic retinopathy. Restoration of beta adrenergic receptor signaling is expected to prevent activation of inflammatory mediators and the histological lesions noted in the retina of the diabetic rat. The studies described herein involve administration of beta adrenergic agonists in topical eye drops, and evaluate the effect of such therapy on retinal pathology and inflammation noted in animal models of diabetes. Additional studies described herein evaluate dose-response curves and the optimal time course for treatment.

Beta-adrenergic agonists are expected to be most effective for treatment because retinal changes observed after sympathectomy can be mimicked by placing an osmotic pump of propranolol, a beta-adrenergic receptor antagonist, in the rat, while phentolamine, an alpha-adrenergic receptor antagonist, had no effect.

Stimulation with a beta-adrenergic receptor agonist could have potentially severe complications if given systemically due to effects on the heart, but the eye offers a unique opportunity to locally deliver drugs. Topical administration to the eye is also preferable for human patients on long-term therapy because it is well tolerated and easily administered.

The data described herein show that when eye drops of isoproterenol, a beta-adrenergic receptor agonist, are administered to animals, the isoproterenol can reach the retina to activate beta-adrenergic receptor signaling cascades there. Activation of such receptors activates cyclic AMP, leading to phosphorylation of protein kinase A, and to phosphorylation of cAMP responsive binding protein (CREB) to activate gene transcription. Isoproterenol therapy was shown to result in increased PKA activity and CREB protein expression in the retina.

A therapeutically effective amount of beta adrenergic agonist is preferably an amount effective to activate a beta adrenergic receptor in the retina. Such an amount is expected to reduce the histological changes commonly noted in pre-proliferative retinopathy. An amount of drug effective to treat retinopathy is an amount that slows, stops or reverses one or more of the histological, chemical or clinical signs or symptoms of retinopathy, including thickening of the basement membrane, loss of pericytes, abnormal proliferation of endothelial cells, neovascularization, the formation of microaneurysms, and activation of inflammatory mediators.

Exemplary beta adrenergic agonists may be in the form of any pharmaceutically acceptable salt and include beta-1, beta-2, or beta-3 receptor agonists, as well as beta adrenergic agonists that are non-selective among the three beta receptors. Studies described herein determine whether activation of the beta-1, -2 or -3 adrenergic receptor is most effective. Nonlimiting examples of beta-1 adrenergic agonists include xamoterol (e.g. hemifumarate salt, also known as ICI 118,587), prenalterol, and denopamine. Nonlimiting examples of beta-2 adrenergic agonists include formoterol (e.g. hemifumarate salt, also known as BD 40A), procaterol hydrochloride, salbutamol (e.g. hemisulfate salt), also known as albuterol, which is not completely selective but is relatively more potent for the beta-2 adrenergic receptor, or salmeterol (also known as GR 33343) which is a potent and long-acting beta-2 adrenergic agonist. Other beta-2 agonists include clenbuterol, levalbuterol, terbutaline, pirbuterol, metaproterenol, fenoterol, bitolterol mesylate, butoxamine, and bambuterol. Nonlimiting examples of beta-3 adrenergic agonists include BRL 37344 (e.g. sodium salt), CL-316243 (e.g., disodium salt), ICI 215,001 (e.g., hydrochloride salt), L-755,507, Pindolol, ZD 2079, or ZD 7114 (e.g., hydrochloride salt), L-796568, and FR165914. Nonlimiting examples of non-selective beta adrenergic agonists include cimaterol, dobutamine (e.g. hydrochloride salt), or isoproterenol (e.g. hydrochloride salt), norepinephrine, and epinephrine.

It is contemplated that the ophthalmic compositions of the invention may be administered in combination with other therapies for treating the underlying disease state. Combination therapy includes administration of the agents together in the same composition, or in different compositions. Administration of the first agent may be at the same time as, or before or after the second agent, e.g. by intervals ranging from minutes to hours, as long as both agents achieve effective concentrations at the site of action or are able to exert their therapeutic effect at overlapping time periods. The two agents may be administered by the same route or different routes, e.g. one agent may be administered topically and the other may administered via intravitreal injection.

While topical administration is most desirable, a variety of modes of administration are possible, including intravitreous or other local injection, including into a depot for long-term release, intraocular or retrobulbar.

Compositions comprising at least 50, 75, 100, 125, 150, 175, 200, 225, 250, 275, 300, 325, 350, 375, 400 ug/mL or a higher concentration of a beta adrenergic agonist such as isoproterenol. A dose of 100 uM (4 drops of 100 uM isoproterenol) was effective at the 8 hour time point. Doses of 1, 2, 3, 4, 5, 6, 7, 8, 9 or 10 drops can be administered twice daily, once daily, on alternative days, twice a week, weekly, every 2 weeks, every 3 weeks, or monthly. Smaller doses may be sufficient when more potent or more selective agonists are used.

It is understood that the suitable dose of a composition according to the present invention will depend upon the age, health and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This typically involves adjustment of a standard dose, e.g., reduction of the dose if the patient has a low body weight.

The frequency of dosing will depend on the pharmacokinetic parameters of the agent and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042), incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

The final dosage regimen will be determined by the attending physician, considering factors which modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

Any pharmaceutically acceptable salt of the beta adrenergic agonist may be used in the ophthalmic compositions of the invention. Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′ dibenzylethylenediamine, chloroprocaine, choline, diethanolamine, dicyclohexylamine, ethylenediamine, N methylglucamine, and procaine.

Pharmaceutically acceptable acid addition salts include inorganic or organic acid salts. Examples of suitable acid salts include the hydrochlorides, acetates, citrates, salicylates, nitrates, phosphates. Other suitable pharmaceutically acceptable salts are well known to those skilled in the art and include, for example, acetic, citric, oxalic, tartaric, or mandelic acids, hydrochloric acid, hydrobromic acid, sulfuric acid or phosphoric acid; with organic carboxylic, sulfonic, sulfo or phospho acids or N substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4 aminosalicylic acid, 2 phenoxybenzoic acid, 2 acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2 hydroxyethanesulfonic acid, ethane 1,2 disulfonic acid, benzenesulfonic acid, 4 methylbenzenesulfoc acid, naphthalene 2 sulfonic acid, naphthalene 1,5 disulfonic acid, 2 or 3 phosphoglycerate, glucose 6 phosphate, N cyclohexylsulfamic acid (with the formation of cyclamates), or with other acid organic compounds, such as ascorbic acid.

Aqueous solutions are generally preferred, based on ease of formulation and administration by dropping into the eye. However, the compositions may also be suspensions, viscous or semi-viscous gels, or other types of solid or semi-solid compositions. Other delivery systems, such as soft contact lenses or delivery as prodrug compounds known in the art are contemplated.

Various tonicity agents may be included in the compositions of the present invention to adjust tonicity, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the compositions will have one or more tonicity agents in a total concentration sufficient to cause the composition to have an osmolality of about 200-400 mOsm.

An appropriate buffer system (e.g., sodium phosphate, sodium acetate, sodium citrate, sodium borate or boric acid) may be added to the compositions to prevent pH drift under storage conditions. The particular concentration will vary, depending on the agent employed. In general, however, the buffering agent will be present in an amount sufficient to hold the pH within the range 6.5-8.0, preferably 6.8-7.6.

A solubilizing or stabilizing agent such as a surfactant can be included to reduce precipitation and increase shelf-life.

An antioxidant may be added to compositions of the present invention to protect from oxidation during storage or use. Examples of such antioxidants include, but are not limited to, vitamin E and analogs thereof, ascorbic acid and derivatives, and butylated hydroxyanisole (BHA).

Ophthalmic solutions typically contain preservatives. Thus, an antimicrobial preservative that kills or inhibits the growth of microbes, e.g. bacteria, fungi, yeast or parasites, may also be added to the compositions of the present invention to prevent or retard microbial growth during storage or use. Nonlimiting examples of preservatives include benzyl alcohol, benzalkonium chloride, phenol, m-cresol, methyl p-hydroxybenzoate, benzoic acid, phenoxyethanol, methyl paraben, and propyl paraben and combinations of any of the above.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice, rats, rabbits, guinea pigs and hamsters; and poultry such as chickens, turkey ducks and geese.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Dopamine Beta-Hydroxylase Expression in the Retina of Diabetic Rats

In this experiment, expression of dopamine beta hydroxlyase, a marker of sympathetic neurotransmission, was measured in the retina of diabetic rats. Three animals were used for each group (normal, 4-week diabetic, and 15-week diabetic rats). All diabetic rats received 60 mg/kg streptozotocin (STZ) on day 1 of the STZ-treatment. Body weights were taken weekly to ensure that animals maintain within 20% of the pre-drug body weight. If greater than 20% of body weight was lost, 1 dose of insulin was given. Normal animals received sham injection of citric acid buffer. At 4 weeks after injection, 3 animals from the 4-week group were sacrificed. At the 15 week time point, the normal and the 15-week STZ animals were sacrificed. Whole eye globes were removed and placed into 4% paraformaldehyde for 24 hours. 5 micrometer sections were then taken from paraffin embedded eyes. Immunohistochemistry was done using a graded alcohol sequence, followed by Proteinase K for antigen retrieval. Washing of the slides was followed by blocking in 1 microgram/ml bovine serum albumin in PBS for 2 hours. Primary antibodies to DBH (1:500, Chemicon) were applied to the slides and slides were placed at 4° C. overnight. The following morning, anti-mouse secondary antibodies conjugated to CY3 were applied for 2 hours at room temperature in the dark. Slides were coverslipped using Fluoromount G and viewed on an Olympus BX50WI microscope with fluorescence. Analyses of slides were done using a Retiga camera with a Macintosh G4 computer and Openlab software. Positive controls were the attached choroid and negative controls were primary antibody omission. All slides were processed simultaneously in the same manner.

The data showed that retinal dopamine β-hydroxylase expression, a marker of sympathetic neurotransmission, was reduced in diabetic rats. Immunoreactivity is much more intense in the outer plexiform layer of the normal rat and comparatively reduced in the diabetic rat. Staining is reduced significantly after only 4 weeks after diabetes onset. These results suggest that diabetes significantly affects the retina's ability to convert dopamine to norepinephrine, indicating a likely reduction in sympathetic neurotransmission in diabetes.

### Example 2

### Effect of Sympathetic Denervation on Histopathology of Retina

In this experiment, 27 female non-diabetic Sprague-Dawley rats were anesthetized and sympathetic innervation to the eye was destroyed by surgical removal of the right superior cervical ganglion. After a period of 6 weeks, basement membrane changes were assessed by real-time PCR to determine expression of two key basement membrane components (laminin-β1 and fibronectin) and electron microscopy to determine basement membrane thickness. The number of pericytes was measured by immunofluorescent staining for NG2 proteoglycan. Steady-state mRNA levels were also evaluated for platelet-derived growth factor-BB (PDGF-BB). Procedures were carried out as described in Wiley et al., Invest Ophthal Vis Sci, 2005. 46:744-748.

Loss of sympathetic innervation caused a significant increase in steady state mRNA levels of fibronectin and a 15% increase in laminin-beta 1 mRNA 3 weeks after surgical sympathectomy. Protein expression also increased at this point. In addition, capillary basement membrane thickness increased significantly.

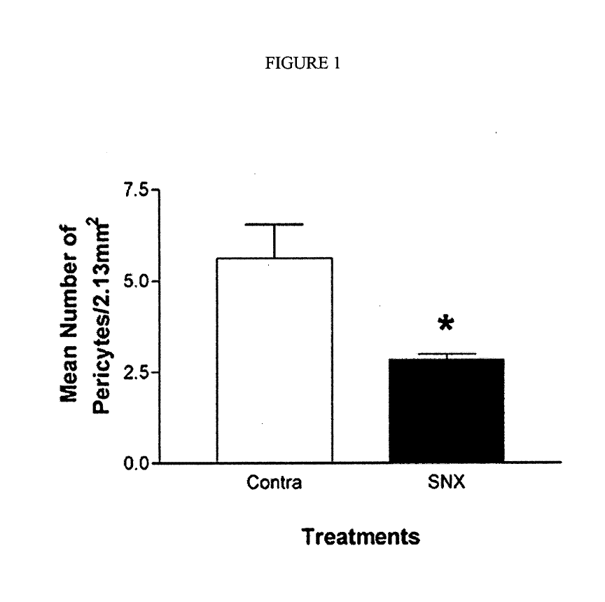

NG2 proteoglycan staining decreased significantly in pericytes in the sympathectomized rat retina. Steady state mRNA for PDGF-BB decreased significantly 6 weeks after surgery. FIG. 1 is a bar graph showing mean numbers of pericytes in contralateral (contra) and sympathectomized (SNX) retina at 6 weeks post-surgery, and demonstrates that significantly fewer pericytes are present after sympathectomy (P<0.05 vs. contralateral, N=4).

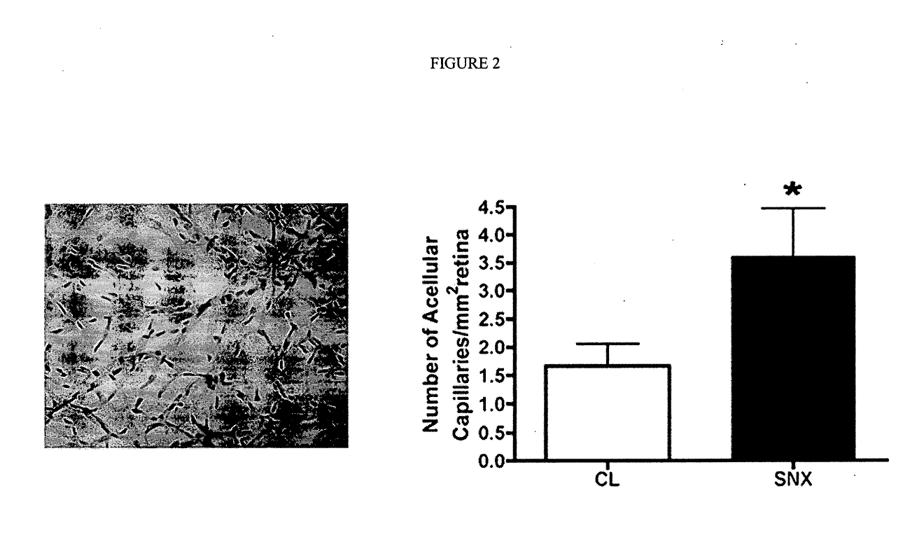

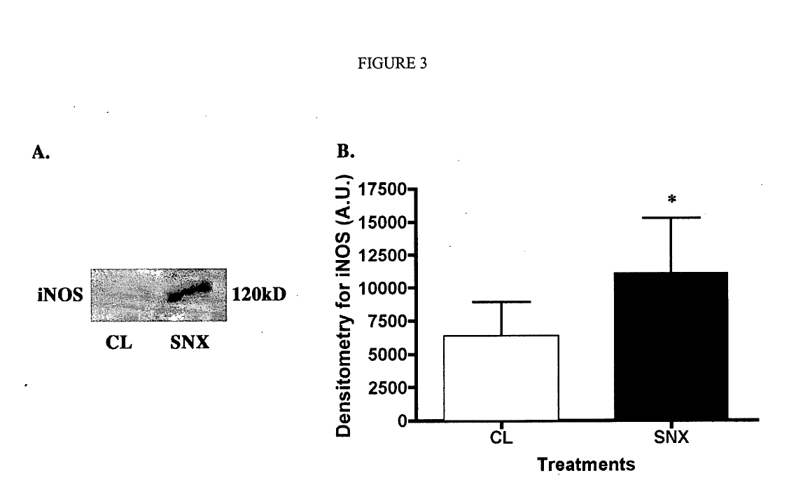

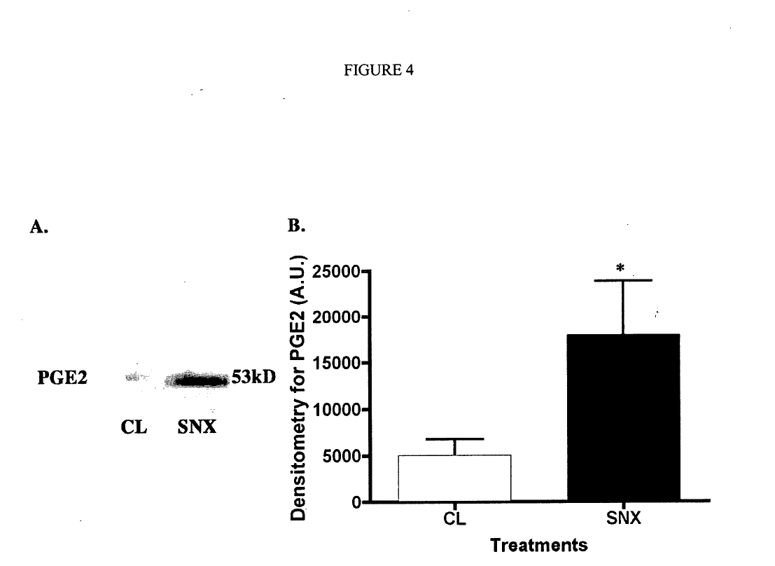

FIG. 2 shows the mean number of acellular capillaries noted in the retina after sympathectomy (SNX) or in the contralateral control (CL), and demonstrates that significantly more acellular capillaries are present after sympathectomy (P<0.05 vs. contralateral, N=4).

The data showed that superior cervical ganglionectomy results in a significant loss of pericytes in the retina and increased thickness of retinal capillary basement membranes. Data also showed that significantly more acellular capillaries are formed in the denervated retina as compared to the contralateral retina of the same rat. Thus, sympathetic denervation results in the types of changes that are hallmarks of pre-proliferative retinopathy in the diabetic rat model.

### Example 3A

### Effect of Sympathetic Denervation on Inflammatory Mediators in Retina

Current work on retinopathy has suggested that some components of the disease may be related to chronic inflammation. These studies determined whether two markers of inflammation, iNOS and PGE2, were increased after sympathectomy. Both iNOS and prostaglandins are known to mediate inflammation and have been implicated in the pathogenesis of diabetic retinopathy (Du et al., Am. J. Physiol. Regul. Integr. Comp. Physiol. 287 (2004) (4), pp. R735-R741).

Studies were carried out as follows on retina of sympathectomized rats to assess mRNA and protein expression of inducible nitric oxide (iNOS), expression levels of PGE2-EP2 receptor subtype, and levels of prostaglandin E2 (PGE2). Female Sprague-Dawley rats weighing 180-200 g (approximately postnatal day 60) were anesthetized by intraperitoneal injection of a mixture of ketamine hydrochloride (27.5 mg/kg, Sanofi Winthrop, New York), xylazine hydrochloride (2.5 mg/kg, Rompun, Miles, Shawnee Mission, Kans.), and atropine sulfate (0.24 mg/kg, Vedco, St. Joseph, Mo.), and an ventral midline incision was made aseptically in the neck. The right superior cervical ganglion was excised by transecting the cervical preganglionic sympathetic nerve and the internal and external carotid nerves; this produces complete and sustained loss of orbital sympathetic innervation in adult rats. The wound was closed with monofilament silk. All rats recovered without signs of distress. Six weeks following surgery, animals were euthanized, and the eyes were removed. The cornea, lens, and aqueous humor were discarded, and the retina of the sympathectomized (SNX) eye and the contralateral (CL) eye were collected.

The mRNA levels for iNOS were assessed as follows. RNA was isolated from the retina using TriReagent® (Molecular Research Center, Inc.). RNA isolation was performed using chloroform and isopropanol. RNA purity was detected by agarose gel electrophoresis, and RNA concentration was measured spectrophotometrically. Reverse transcription of 1 μg RNA for cDNA synthesis was carried out using an Improm II Kit (Promega, Madison, Wis.). The reaction mixture (DEPC water, Improm II 5× reaction buffer, 25 mM MgCl₂, 10 mM dNTP, and 20 Units RNAsin, and 1 μM oligo dT) and 1 μg RNA were extended for 60 min at 42° C., followed by heat-inactivation of the reverse transcriptase enzyme at 70° C. for 15 min. RNase A inhibitor (0.2 μL, 10 mg/ml) was added, followed by incubation for 30 min at 37° C. Samples were stored at −20° C. for real-time PCR. Real-time PCR was done to detect rat iNOS and GAPDH mRNA levels as described previously (Steinle and Lashbrook, Exp. Eye Res. 83 (2006) (1), pp. 16-23 and Wiley et al., Invest. Ophthalmol. Vis. Sci. 46 (2005) (2), pp. 744-748). PCR primers were designed using the GCG Software Prime and were chosen to generate an amplicon smaller than 200 base pairs. GAPDH was used as a control housekeeping gene, as it is expressed in all tissues and is not altered following sympathectomy. Significance in the (2-ΔΔCT) between the sympathectomized and contralateral rat retina were analyzed using a paired T-test from Prism Software (GraphPad, San Diego, Calif.), with significance accepted at P<0.05.

Protein expression of PGE2-EP2 receptor subtype was assessed by Western blot as follows. Retina from the sympathectomized (SNX) eye and the contralateral (CL) eye were placed into cold lysis buffer (50 mM Tris-HCl, pH 7.4; 1% NP-40, 0.25% Na-deoxycholate; 150 mM NaCl; 1 mM EDTA; 1 mM PMSF; 1 μg/ml each of aprotinin, leupeptin, pepstatin; 1 mM Na₃VO₄; 1 mM NaF; 0.1% SDS) for homogenization. Retinal samples were assayed for protein content and then stored at −80° C.

Denaturing sample buffer (1 ml 2×GDW, 640 μl 1 M Tris-HCl, pH 6.8, 420 μl 30% glycerol, 250 μl β-mercaptoethanol, 200 μL 0.05% bromophenol blue, and 0.125 g recrystallized SDS) was added to 50 μg of protein, as determined by Bradford assay. Protein samples with sample buffer were centrifuged for 5 min to thoroughly mix the samples, followed by heat denaturation for 5 min at 100° C. Protein samples were then separated on 4-12% pre-cast tris-glycine gels (Invitrogen, Carlsbad, Calif.). Electrophoresis and immunoblotting were done as previously described (Steinle et al., J. Biol. Chem. 278 (2003) (23), pp. 20681-20686) using antibodies to iNOS (diluted 1:500, Chemicon, Temecula, Calif.) and PGE2-EP2 receptor subtype (diluted 1:500, Chemicon). Mean densitometry of immunoreactive bands was assessed using Kodak software, and results are expressed in densitometric units. A paired T-test was performed between the sympathectomized and contralateral retina with significance accepted at P<0.05.

An ELISA assay to measure PGE2 content in cell culture supernatants and retinal samples was purchased from Endogen (Pierce Biotechnology, Rockford, Ill.) and used according to the manufacturer's instructions.

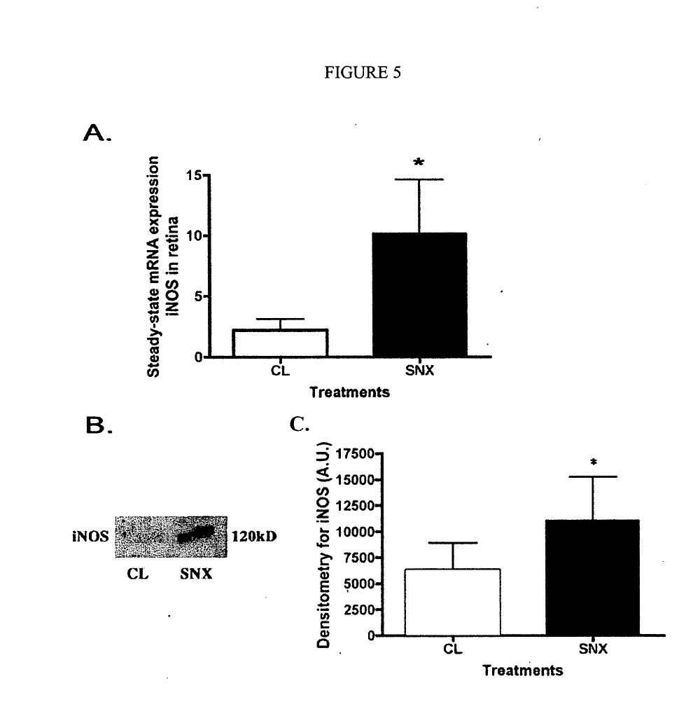

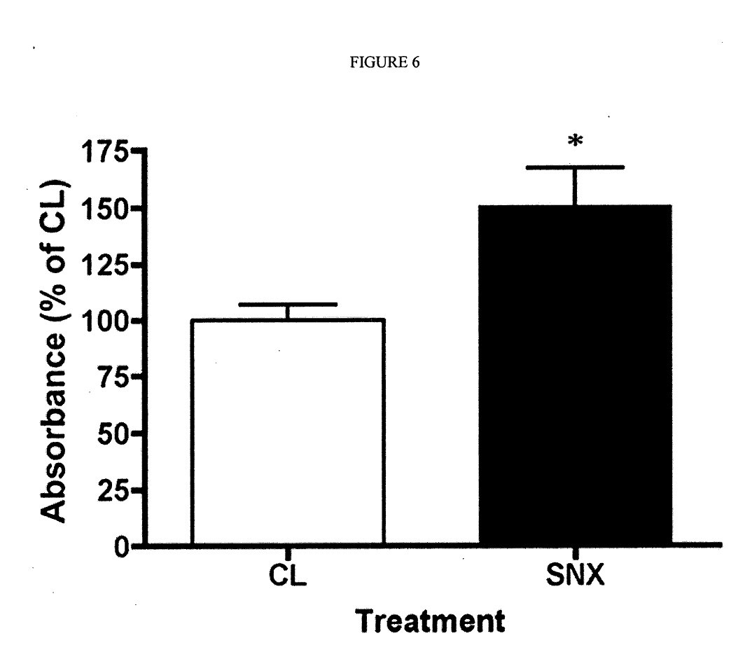

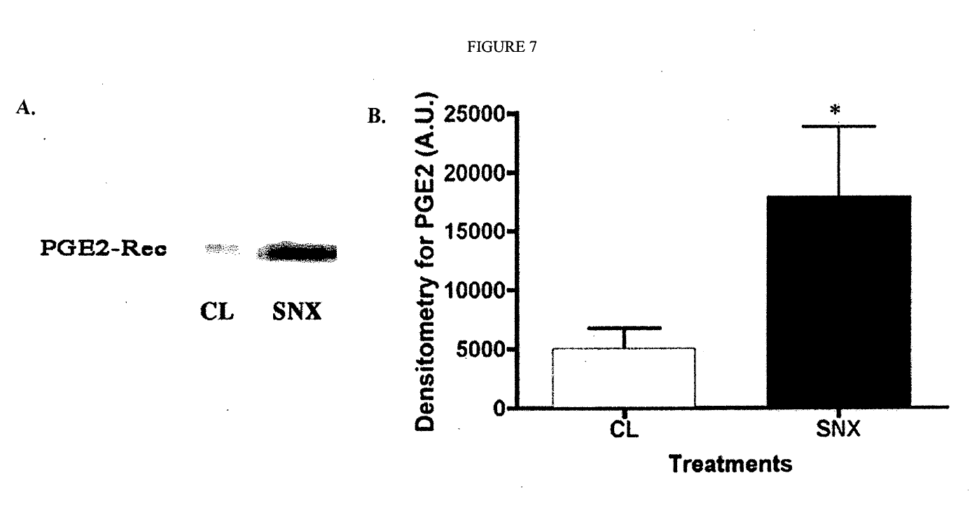

Representative results of these mRNA and protein assays are shown in FIGS. 5-7. Six weeks following surgical removal of the superior cervical ganglion, steady-state mRNA expression of iNOS is significantly increased in the retina (P<0.05, FIG. 5A). Additionally, protein expression for iNOS is also upregulated in the sympathectomized retina as compared to the contralateral (P<0.05, FIGS. 5B and 5C), suggesting that sympathetic neurotransmission is modulating iNOS expression. Levels of PGE2 were significantly increased following sympathectomy as compared to the contralateral retina (P<0.05, FIG. 6), and PGE2-EP2 receptor protein expression was significantly increased as well (P<0.05, FIGS. 7A and 7B). This significant increase in PGE2-EP2 receptor and PGE2 levels indicates that PGE2 is also under control of sympathetic neurotransmission in the retina.

### Example 3B

### Effect of Beta-Adrenergic Agonists on Inflammatory Mediators in the Retina

To evaluate the effect of β-adrenergic agonists in modulation of inflammatory markers, β-adrenergic agonists are applied to a number of different cell types in the human retina that are known to produce inflammatory mediators. Retinal endothelial cells have been shown to produce both endothelial NOS (eNOS) and iNOS (Chakravarthy et al., Current Eye Research 14 (1995) (4), pp. 285-294). Increased expression of eNOS has been reported in the retina of rats exposed to hypoxia (Kaur et al., Invest. Ophthalmol. Vis. Sci. 47 (2006) (3), pp. 1126-1141); while others have reported that iNOS expression can be activated in retinal endothelial cells (Chakravarthy et al., supra). In addition to retinal endothelial cells, retinal pericytes may also show iNOS activity when stimulated (Chakravarthy et al., supra) or in cells exposed to altered glucose levels (Kim et al., Exp. Eye Res. 81 (2005) (1), pp. 65-70). In patients with diabetes, iNOS immunoreactivity was noted in the ganglion cells and inner cell layer of the retina and in glial cells (Abu E1-Asrar et al., Eye (London, England) 18 (2004) (3), pp. 306-313). In cells cultured in normoxia or hypoxia, only retinal glial cells increased nitrite production (Kashiwagi et al., Brain Res. Mol. Brain Res. 112 (2003) (1-2), pp. 126-134).

Initial studies focused on human retinal endothelial cells, which express β-adrenergic receptors. Human microvascular retinal endothelial cells were isolated by Applied Cell Biology Research Institute and sold by Cell Systems (Kirkland, Wash.). Cells were used at passage 3-5 for all experiments. Cells were identified as endothelial cells on the basis of their cobblestone morphology. Cells were grown in attachment factor-coated Corning® dishes and in either high or low glucose media. Both high and low glucose media were identical (purchased from Cell Systems and supplemented with 20% fetal bovine serum, 100 IU/ml penicillin, 100 μg/ml streptomycin, and 0.25 μg/ml amphotericin B) except for the glucose concentration: 25 mM glucose (high glc) or 5 mM glucose (low glc). Cells were adjusted to the appropriate glucose environment for 5 days before the experiments were conducted in starvation medium. Starvation medium had the appropriate concentration of glucose and contains all of the above ingredients except that 0.2% bovine serum albumin is substituted for 20% serum.

For experiments, cells were starved for 18-24 h using starvation medium to insure that the results obtained are due to agonist stimulation, not residual effects from the serum. Following starvation, 10 μM isoproterenol (non-specific β-adrenergic receptor agonist), 10 μM xamoterol (β1-adrenergic receptor subtype agonist), or 10 μM BRL37344 (β3-adrenergic receptor subtype agonist) was placed onto dishes and allowed to act on the cells for 1, 2, or 6 h. Three dishes received no treatment and serve as not-treated controls. At 1, 2, or 6 h following agonist treatment in either low or high glucose, medium was aspirated, cells were washed, and cold lysis buffer was applied to the cells. The effects of xamoterol and BRL37344 were evaluated under high glucose conditions only.

Western blot analysis of iNOS and PGE2 expression was done as described in Example 5A for animal retina, except that cold lysis buffer was placed into the culture dishes, and endothelial cells were scraped into centrifuge vials. Western blot analysis methods were similar to that described previously except that a one-way ANOVA or unpaired T-test (two-tailed) was used for statistics between the treatment groups with significance accepted at P<0.05.

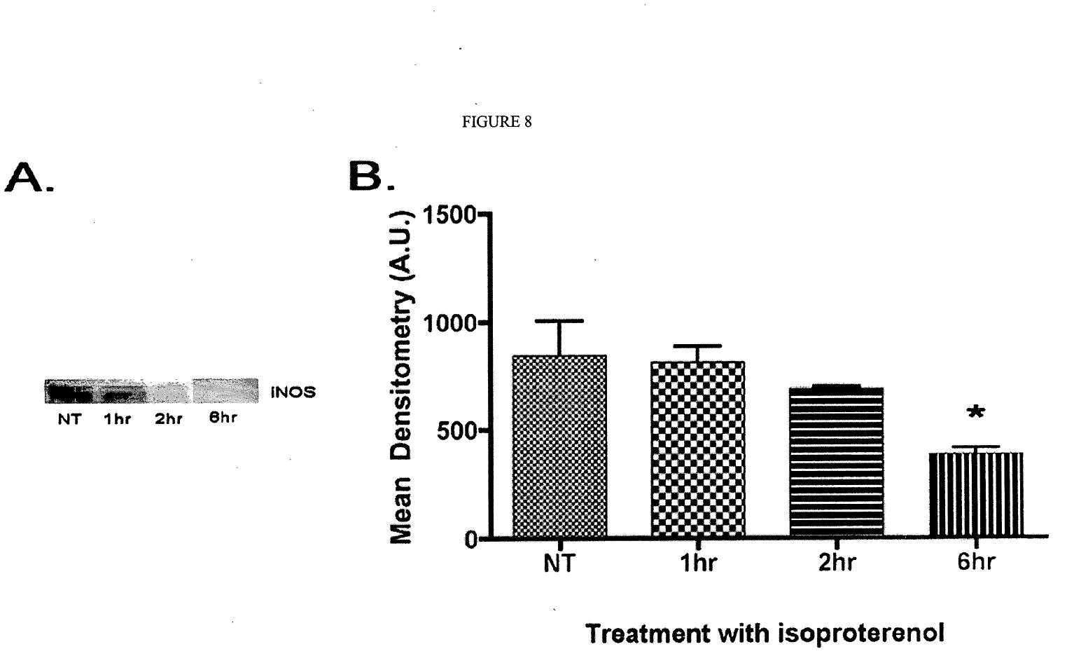

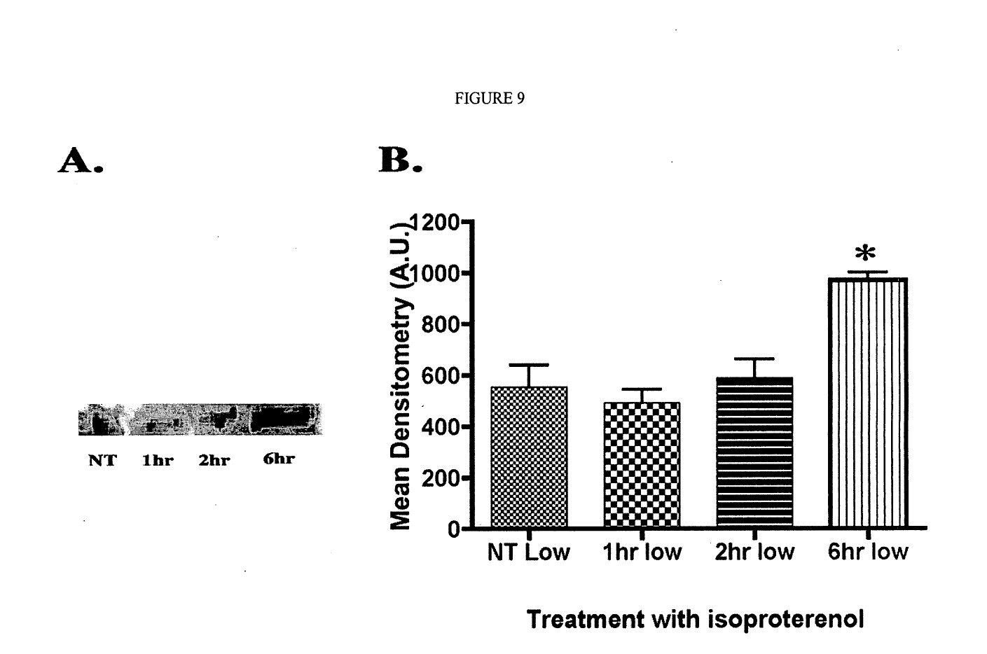

In the high glucose medium, treatment with 10 μM isoproterenol 6 h prior to cell collection significantly reduced iNOS protein expression (P<0.05 vs. not treated, FIG. 8A,B). In retinal endothelial cells exposed to the low glucose medium, isoproterenol resulted in an upregulation of iNOS protein following 6 h of isoproterenol stimulation (P<0.05 vs. not treated, FIG. 9A,B), indicating that isoproterenol is effective at reducing iNOS protein expression in a hyperglycemic environment, but not in normal glucose medium.

FIG. 8A shows a typical western blot for iNOS protein expression in human retinal endothelial cells grown in high glucose (25 mM glucose) medium and either not-treated (NT) or treated with 10 μM isoproterenol for 1 h (1 hr), 2 h (2 hr) or 6 h (6 hr). FIG. 8B shows the mean densitometry of all dishes at each time point. Each time point was investigated in three independent experiments. *P<0.05 vs. not-treated.

FIG. 9A shows a typical western blot for iNOS protein expression in human retinal endothelial cells grown in low glucose (5 mM glucose) medium and either not-treated (NT) or treated with 10 μM isoproterenol for 1 h (1 hr), 2 h (2 hr), or 6 h (6 hr). FIG. 9B shows mean densitometry of all dishes at each time point. Each time point was investigated in three independent experiments. *P<0.05 vs. all other time points investigated.

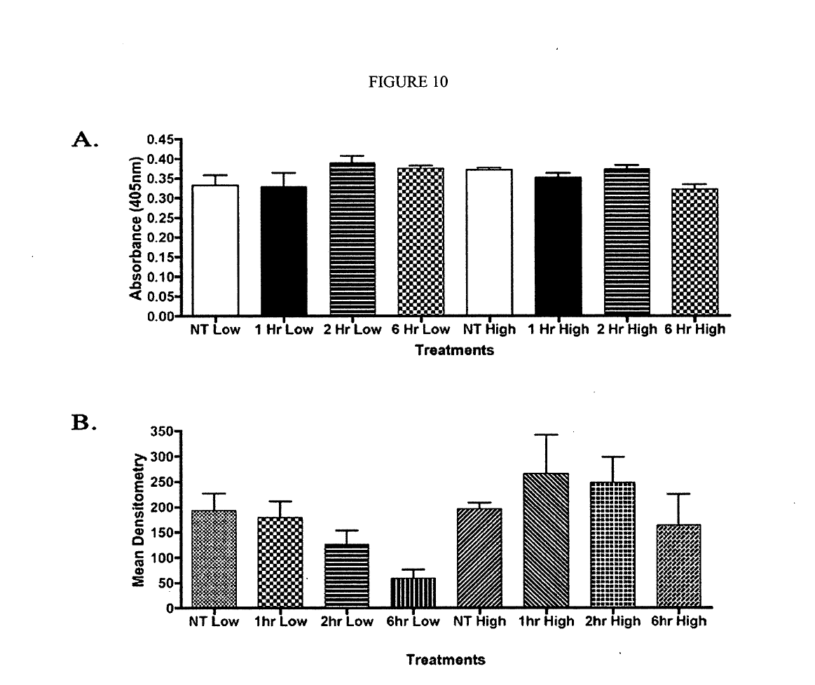

Treatment with the isoproterenol in either low or high glucose media did not affect PGE2 protein levels (FIG. 10A) or PGE2-EP2 receptor subtype expression (FIG. 10B) in human retinal endothelial cells. The length of isoproterenol treatment did not alter PGE2-EP2 receptor protein expression in either low or high glucose medium, suggesting that retinal endothelial cells are not involved in adrenergic receptor modulation of PGE2 protein and receptor levels.

FIG. 10A shows absorbance values for PGE2 levels in human retinal endothelial cells exposed to either high (25 mM, High) or low (5 mM, Low) glucose medium and not-treated (NT) or treated with isoproterenol for 1 h (1 hr), 2 h (2 hr), or 6 h (6 hr). FIG. 10B shows mean densitometry for PGE2 receptor expression in human retinal endothelial cells exposed to either high (25 mM, High) or low (5 mM, Low) glucose medium and not-treated (NT) or treated with isoproterenol for 1 h (1 hr), 2 h (2 hr), or 6 h (6 hr). Each time point was investigated in three independent experiments.

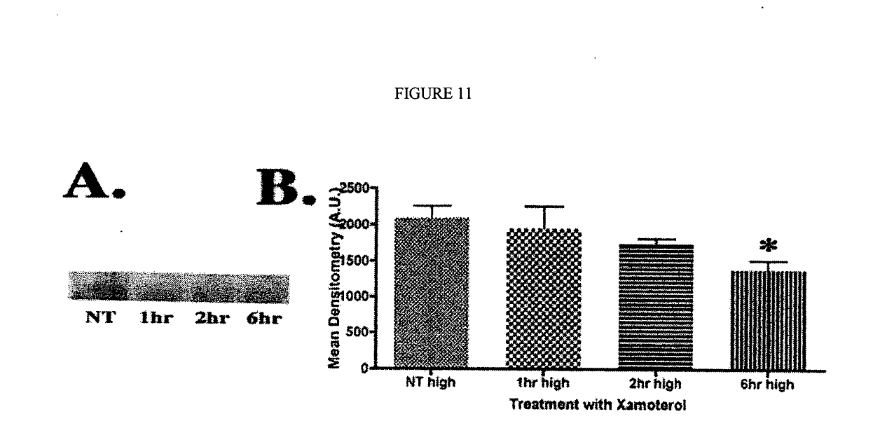

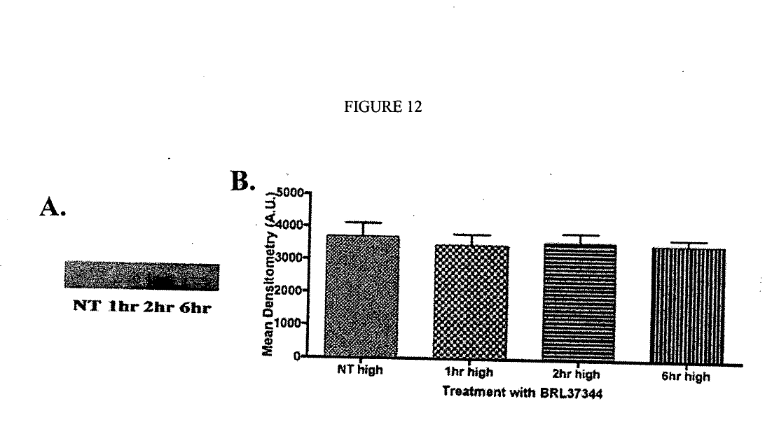

Treatment with xamoterol, a beta-1 adrenergic agonist, was observed to decrease iNOS protein expression, while BRL37344 had no effect on iNOS expression in human retinal endothelial cells. Results shown in FIG. 11 indicate that predominantly β1-adrenergic receptors are involved, as stimulation with xamoterol produced decreased iNOS protein expression (P<0.05 vs. NT) in a manner similar to isoproterenol. BRL37344 had no effect on iNOS protein expression (FIG. 12B).

FIG. 11A shows a typical western blot and FIG. 11B shows mean densitometry for iNOS expression in human retinal endothelial cells exposed to high glucose medium and not-treated (NT) or treated with 10 μM xamoterol for 1 h (1 hr), 2 h (2 hr), or 6 h (6 hr). FIG. 12A shows a typical western blot and FIG. 12B shows mean densitometry for iNOS expression in human retinal endothelial cells exposed to high glucose medium and not-treated (NT) or treated with 10 μM BRL37344 for 1 h (1 hr), 2 h (2 hr), or 6 h (6 hr). Each time point was investigated in three independent experiments. *P<0.05 vs. NT.

Thus, the results showed that when adrenergic signaling was eliminated through sympathectomy, a significant increase in gene and protein expression of iNOS, increased PGE2 levels, and increased protein expression of the PGE2 receptor subtype EP2 are noted. The increased expression of iNOS appears to be regulated by β1-adrenergic receptors, as isoproterenol and xamoterol can modulate its expression in human retinal endothelial cell cultured in high glucose environments. Retinal endothelial cells do not appear to play a significant role in the changes in PGE2 levels and PGE2-EP2 receptor expression after sympathectomy. Similar experiments are conducted with other cell types in the retina, such as pericytes, glial cells or Müller cells (which also express β-adrenergic receptors) to confirm effects in these other cell types.

### Example 4

### Effect of Topical Ophthalmic Administration of Beta Adrenergic Agonists on Retina

This study determined whether replacement of beta-adrenergic receptor signaling in the retina was feasible in an eye drop form. Stimulation of beta adrenergic receptors initially results in increased PKA activity; the next step in intracellular signaling following increased PKA activity is an increase in cAMP responsive binding element protein (CREB).

Three groups of animals were used for these experiments. The first group was made diabetic by a single injection of 60 mg/kg streptozotocin and allowed to remain diabetic for 2.5 month and received 4 drops of 100 μM isoproterenol placed into both eyes (STZ). The other two groups were control groups, with one group being non-diabetic but receiving the 100 μM isoproterenol eye drops (Normal), and the other group not receiving any treatment (NT). At 4, 8 and 12 hours after treatment with isoproterenol, retina from the rats was assayed for PKA by ELISA and CREB by Western blot analysis.

The retina of rats from each group was assayed for PKA by ELISA. Three rats were evaluated at each time point (4 hours, 8 hours and 12 hours). The PKA ELISA was carried out using the MESACUP Protein Kinase Assay System from Upstate (Lake Placid, N.Y.). To begin the experiments, 0.1M ATP was made and set to a pH of 7.0. cAMP at a concentration of 20 μM was made with reagents purchased from Sigma Aldrich. To obtain the samples to be assayed, animals were sacrificed and the retina was removed and placed in reaction buffer supplied in the kit. One hundred microliters of each sample in reaction buffer was placed into coated wells of a 96-well plate in duplicate. Samples were incubated at 27° C. for 20 minutes, followed by the addition of 100 μL of stop solution (meant to stop reaction of samples with kinase in the component mixture coating of the plate). Wells were washed thoroughly 5 times and then 100 μL of the biotinylated antibody was added for 60 minutes at 27° C. Following washing again for 5 times, the POD-conjugated streptavidin was added, and plate was placed at 27° C. for 1 hour. After thorough washing, 100 μL of substrate solution was added to the wells and allowed to incubate at 27° C. for 5 minutes. Following application of 100 μL of stop solution to end the reaction with the substrate, the plate was read at 490 nm on a BioTek Plate Reader.

FIG. 5 shows the results of PKA ELISA performed on retinal lysates from these three groups of rats: not-treated, normal treated (non-diabetic and treated with eye drops), and diabetic treated (diabetic for 2.5 months and treated with eye drops). The results demonstrate that the isoproterenol eye drops significantly increased PKA activity as measured by ELISA (Upstate, Lake Placid, N.Y.) at the 8-hour time point (*P<0.05 vs. not-treated, #P<0.05 vs. normal, N=3). The hatched bars in the figure are treated groups. PKA was also significantly increased at 12 hours after treatment relative to the normal, but not when compared to the not treated group.

Western blot analysis was also done to determine protein expression of CREB. Two rats were evaluated at each time point (4 hours, 8 hours and 12 hours). Animals were sacrificed and the eyes were removed. The retina was extracted and placed into lysis buffer (50 mM Tris-HCL, pH 7.4; 1% NP-40, 0.25% Na-deoxycholate; 150 mM NaCl; 1 mM EDTA; 1 mM PMSF; 1 μg/ml each of aprotinin, leupeptin, pepstatin; 1 mM Na₃VO₄; 1 mM NaF; 0.1% SDS). Protein lysates were homogenized and centrifuged to generate a pellet and supernatant. The supernatant was used for a Bradford protein assay to determine protein concentrations in the samples. Once protein concentrations were known, denaturing sample buffer (1 mL 2×GDW, 640 μL 1M Tris-HCL pH 6.8, 420 μL 30% glycerol, 250 μL beta-mercaptoethanol, 200 μL 0.05% bromophenol blue, and 0.125 g recrystallized SDS) was added to 30 μg of protein. Protein samples were separated on 4-12% pre-cast tris-glycine gels (Invitrogen, Carlsbad, Calif.). Gels were run at 130V for one and a half hours and then blotted onto a nitrocellulose membrane at 30V for one and a half hours. For antibody detection, the membrane was blocked overnight at 4° C. in block buffer (1 mM Tris pH 7.5, 150 mM NaCl, and 0.05% Tween) with 5% dry milk. Primary polyclonal antibodies to CREB (1:500, Cell Signaling) were applied for 2 hours at room temperature. Membranes were probed with horseradish peroxidase-conjugated anti-rabbit secondary antibodies applied at a 1:5000 dilution at room temperature for two hours. Immunoreactive bands were detected by enhanced SuperSignal (Pierce, Rockford, Ill.) and analyzed using the Kodak 2000R image station. Mean densitometry was assessed using Kodak software, and results are expressed as a percentage of the not treated eyes. A I-way ANOVA was used to compare the time points of STZ and normals versus the not-treated samples, with P<0.05 being accepted as significant.

FIG. 6 shows the results of Western blot analysis of protein expression for CREB. CREB protein expression follows the same pattern as that of PKA activity after application of 100 μM isoproterenol eye drops. Maximal activity was noted at 8 hours after treatment. N=2 for all groups, except not-treated which had N=3. The hatched bars in the figure represent data from groups receiving treatment. The data demonstrate that there was a substantial increase in the CREB expression in diabetic animals that received the isoproterenol eye drops at 8 hours, as compared to the normal group.

Thus, these results showed that treatment of diabetic rats with 4 eye drops of 100 μM isoproterenol significantly increased PKA activity and CREB protein expression in the retina. These results demonstrate the feasibility of administration of beta adrenergic agonists using eye drops, because the isoproterenol was able to reach the retina and initiate cellular signaling characteristic of beta adrenergic receptor activation.

### Example 5

### Beta Adrenergic Receptor Expression in Diabetic Rat Retina

Experiments will be conducted on 48 male rats at 1, 2, 4, and 6 weeks after diabetes onset. These time periods are chosen to coincide with the loss of dopamine beta hydroxylase immunoreactivity in the retina after diabetes onset. For the STZ-treatment, rats will receive 1 subcutaneous injection of 60 mg/kg streptozotocin dissolved in citric acid buffer. Control rats will receive an injection of citric acid buffer alone. Animals will be weighed weekly and glucose measurements taken via the tail vein. Only rats with glucose measurements >250 mg/dl will be used for experiments.

For the real-time PCR experiments, 6 diabetic and 6 control rats will be used at each time point. Rats will be anesthetized using 150 mg/kg pentobarbital and the eye will be removed. The cornea and lens will be discarded, and the retina will be isolated from the posterior uvea and placed into tubes containing TriReagent (Molecular Research Center, Inc.). RNA will be isolated using a double ethanol extraction. Reverse transcriptase and real-time PCR will be done using SYBR green. Primers will be designed to detect each of the β-adrenergic receptor subtypes.

To investigate protein expression of each β-adrenergic receptor subtype, western blot analysis will be used. The other eye from the 6 diabetic and 6 control rats used for real-time PCR will be used for these experiments. The retina will be isolated as above, with the retina being placed directly into protein lysis buffer (1 mM Tris-HCl, pH 7.4; 10 ml 10% Igepal-40; 2.5 ml 10% Na-deoxycholate; 1 ml 100 mM EDTA). Western blot analysis will be done using primary antibodies to each of the β-adrenergic receptor subtypes and densitometry completed using Kodak software. Unpaired two-tailed T-tests will be used to compare the bands between the normal and diabetic retina for each receptor subtype at each time point. One-way ANOVA will be used to compare expression between the time points. Statistics will be completed using the software program Prism (GraphPad, San Diego, Calif.).

If diabetes reduces sympathetic neurotransmission as has been reported by others, reduced gene and protein expression of β-adrenergic receptor subtypes should be observed. A selective reduction in β-1, -2 or -3 adrenergic receptor subtype will indicate that an agonist selective for that type of receptor should be used.

### Example 6

### Effect of Both Sympathetic Denervation and Diabetes on Retina

Ninety-six animals will be used for these experiments at 2, 4, and 6 months after diabetes onset. Forty-eight rats will undergo surgical sympathectomy under ketamine/xylazine/atropine anesthesia, removing the right superior cervical ganglion. After a period of two weeks for wound healing, the 48 sympathectomized rats will receive an injection of 60 mg/kg of streptozotocin dissolved in citric acid buffer. The other 48 rats will receive an injection of citric acid buffer and remain as normal control rats. Insulin will only be given to rats undergoing a 20% reduction of body weight, and only 1-2 Units of insulin will be given as needed to maintain body weight without a loss of the hyperglycemic state. Thirty-two animals will be sacrificed at each time point of 2, 4, and 6 months after the diabetes onset under pentobarbital anesthesia (150 mg/kg). Eight diabetic and 8 normal rats will be used to assess acellular capillary numbers, while the additional 16 rats (8 diabetic, 8 normal) will be used for immunofluorescence for pericyte loss.

Acellular capillary numbers will be assessed using the trypsin digest method. Briefly, the whole globe will be placed into 10% formalin for a minimum of 24 hours. The cornea and lens will be removed, and the retina will be carefully separated from the remaining uvea. The following day, trypsin will be used to remove the vitreous from the retina. The retina will be cleaned using a sable brush, and once clean of all cells, retinal capillaries will be flat-mounted onto a fresh slide and stained with periodic acid-Schiff and hematoxylin. Retinal cells and acellular capillaries will be measured in a masked manner. The number of acellular capillaries will be counted in multiple mid-retinal fields (one field adjacent to each of the 5-7 retinal arterioles radiating from the optic disc) at a magnification of 400×. Acellular capillaries will be scored as those that do not possess a pericyte or endothelial cell along the entire capillary, but are at least 20% of the thickness of neighboring capillaries and 50 μm in length. Statistics will be done to compare the number of acellular capillaries in the sympathectomized and diabetic, the contralateral diabetic, and the normal retina at each time point using a 1-way ANOVA with student Newman Keul's post hoc test.

Pericyte ghosts can be assessed using the same capillary flatmounts as the acellular capillaries. The number of pericyte ghosts observed/1000 capillary cells will be noted. In addition, the remaining 48 eyes (8 diabetic and 8 normal at each of 3 time points) will be placed into 4% paraformaldehyde overnight. Immunofluorescence for NG2 proteoglycan to indicate pericytes will be done. The mean number of pericytes from 10 images/slide for each animal will be determined for each of the groups, sympathectomized and diabetic, contralateral and diabetic, and normal. Means for 8 animals will be compared using a 1-way ANOVA using Prism software. Comparisons will also be made for each group between the time points assessed.

Substantially more acellular capillaries and pericyte ghosts are expected to be observed earlier in the sympathectomized diabetic than in the contralateral diabetic normal retina. The contralateral diabetic retina should have more lesions than the normal, non-diabetic animals. These results would suggest that sympathetic neurotransmission is critical to formation and modulation of the histological lesions observed in the diabetic rat.

### Example 7

### Effect of Beta Adrenergic Agonists on Inflammatory Mediators in Retinal Cells

A retinal Müller cell line or another retinal cell line that expresses beta-adrenergic receptors will be used and cultured using DMEM and 10% FBS. Once cells are confluent, they will be starved for 18-24 hours using DMEM medium with only 0.2% BSA. Following starvation, the cells will be treated with 10 μM isoproterenol for 6, 12, 18, or 24 hours. Some dishes will serve as non-treated controls. Cells will be collected at each of the 4 time points and pelleted in TriReagent for RNA studies or into lysis buffer for western blot or reaction buffer provided in the ELISA kit. Real-time PCR and western blot analysis for iNOS will be done, with primers designed specific for IL-1β, TNFα and iNOS. Primary antibodies to iNOS will be obtained from Chemicon (Temecula, Calif.). ELISA assays for IL-1β, TNFα will be done according to supplied protocols (Chemicon).

Stimulation of beta-adrenergic receptors in culture should reduce expression of IL-1β, TNFα and iNOS. IL-1β and TNFα may have different time courses for decreased gene and protein expression. However, most changes should occur within 24 hours of treatment.

### Example 8

### Effect of Beta Adrenergic Agonists on Inflammatory Mediators in Diabetic Rat Retina

These experiments will be conducted on RNA and protein samples from 8 diabetic rats receiving isoproterenol eye drops at the optimized dose and time course determined in Example 9 below. Eight diabetic rats without eye drops will be used for measuring levels in diabetes and 8 animals treated only with citric acid buffer will serve as controls. At the time point determined to increase PKA activity and CREB expression, animals will be sacrificed using pentobarbital anesthesia. One retina from each animal will be placed into TriReagent for real-time PCR, while the remaining retina will be placed into protein lysis buffer for western blot analysis for iNOS or ELISA reaction buffer for TNFα and IL-1β assays. Real-time PCR, western blotting, and ELISA assays will be done as described above. Gene and protein activity values for each inflammatory marker will be compared using a 1-way ANOVA between the diabetic eye drop, diabetic without eye drop, and no treatment groups.

At the optimized dose and time course, isoproterenol eye drops should decrease gene and protein activity of IL-1β, TNFα and iNOS in the diabetic rats to levels similar to that in rats treated only with citric acid buffer. Expression should be substantially reduced from that noted in the diabetic without eye drop rats. Such results would indicate that isoproterenol therapy, once optimized, can reduce inflammation in the retina, as well as prevent acellular capillary formation and pericyte dropout.

### Example 9

### Pharmacokinetics of Topical Application of Beta Adrenergic Agonists to Eye

These experiments will be conducted on 3 groups of animals at all time points and doses investigated. One group of animals will be diabetic and will receive the isoproterenol therapy, the second group with be diabetic, but will not receive the eye drops, and the third group will be citric acid treated without eye drops. Each group will have 6 animals at each time point and dose used.

For the dose-response experiments, 18 rats will be used at 100 μM, 1 mM, 10 mM and 100 mM of isoproterenol therapy. Time course experiments will employ 18 rats at 6, 8, 12, 18, and 24-hour intervals. Four eye drops will be placed onto each eye of the isoproterenol treated animals under isofluorane anesthesia. At each dose and at the appropriate time course, all 18 animals will be sacrificed under pentobarbital anesthesia. The retina from one eye will be placed into the reaction buffer, supplied in the PKA ELISA kit. The retina from the other eye will be immediately placed into protein lysis buffer. Western blot analysis for CREB will be done, with the primary antibody to phosphorylated CREB used at a dilution of 1:500 and purchased from Cell Signaling. The retina in reaction buffer will be processed for the PKA ELISA according to the manufacturer's instructions (Upstate, Lake Placid, N.Y.). The heart will be removed from the animal and sectioned to look for evidence of hypertrophy, and blood pressure will be measured to exclude a hypertensive effect from systemic absorption of the eyedrops.

It is expected that doses greater than 100 μM can be used without adverse effects. This may allow treatments to occur every 12 or potentially 24 hours.

### Example 10

### Therapeutic Effects of Topical Application of Beta Adrenergic Agonists to Eye

Using the optimized dose and time course for eye drop treatment found in Example 9, 18 rats in 3 groups (6 rats/group) will be used for these experiments at 4, 6, 8, 12 months after diabetes onset. Six animals will be diabetic and will receive isoproterenol eye drop therapy, 6 rats will be diabetic with no eye drop therapy, and 6 citric acid buffer treated rats will be used as controls. At 4, 6, 8, and 12 months after diabetes onset, 18 animals (6 from each treatment group) will be sacrificed under pentobarbital anesthesia. One eye from each animal will be used for trypsin digest to assess acellular capillary formation. The remaining eye will be processed for immunofluorescence for NG2 proteoglycan for measurement of pericyte numbers. Statistics will be done at each time point for acellular capillary counts and pericyte numbers between the groups using a 1-way ANOVA with a post-hoc Student Newman Keul's test.

The heart will also be removed from all animals for staining with hematoxylin and eosin to look for ventricular hypertrophy. Animals will be monitored weekly for weight, and rats showing an arrhythmic heart rate will be sacrificed.

Fewer acellular capillaries and pericyte ghosts are expected to be present in diabetic animals that receive isoproterenol eye drop therapy as compared to the diabetic without eye drop group. It is anticipated that acellular capillary and pericyte counts will be reduced to those similar to the normal, non-diabetic rats. If the heart rate becomes arrhythmic on excessive numbers of animals, the dose or time course for isoproterenol treatments may have to be adjusted to lower or less frequent doses. It may be that the dose and time required to activate PKA activity and CREB protein expression is not sufficient to prevent acellular capillary formation. If it appears that some changes are noted but not reaching statistical significance, doses and/or time courses may need to be adjusted to for higher or more frequent doses.

What is claimed is:

1. A topical ophthalmic composition comprising an amount of beta adrenergic agonist effective to treat retinopathy.

2. The composition of claim 1 further comprising a preservative.

3. The composition of claim 1 further comprising an antioxidant.

4. The composition of claim 1 wherein the beta adrenergic agonist is cimaterol, dobutamine or isoproterenol, or a pharmaceutically acceptable salt thereof.

5. The composition of claim 1 wherein the beta adrenergic agonist is isoproterenol.

6. A method of using the composition of claim 1 to reduce basement membrane thickening, pericyte loss or acellular capillaries in the retina.

7. A method of using the composition of claim 1 to reduce neovascularization in the retina.

8. A method of using the composition of claim 1 to reduce inflammatory mediators in the retina.

9. A method of using the composition of claim 1 to treat diabetic retinopathy.

10. A method of using the composition of claim 1 to treat macular degeneration.
